(19) United States
(12) Patent Application Publication
(10) Pub. No.: US 2025/0255801 A1
(43) Pub. Date: Aug. 14, 2025

(54) Novel Parenteral Composition Comprising Linagliptin or its Salts

(71) Applicant: Zenvision Pharma LLP, Mumbai (IN)

(72) Inventors: Bobba Venkata Sivakumar, Navi Mumbai (IN); Girish Kumar Jain, Navi Mumbai (IN); Shankar Pol, Navi Mumbai (IN); Sunil Pophale, Navi Mumbai (IN)

(21) Appl. No.: 18/701,310

(22) PCT Filed: Oct. 17, 2022
(86) PCT No.: PCT/IN2022/050926

§ 371 Date: Apr. 15, 2024

(30) Foreign Application Priority Data
Oct. 17, 2021 (IN) 202121042172

(51) Int. Cl. A61K 9/00; A61K 9/08; A61K 31/522; A61K 47/02; A61K 47/10; A61K 47/26

(52) U.S. Cl. CPC A61K 9/0019; A61K 9/08; A61K 31/522; A61K 47/02; A61K 47/10; A61K 47/26

(57) ABSTRACT

The present invention relates to a parenteral composition of Linagliptin or its salt and one or more pharmaceutically acceptable excipient. The parenteral composition of Linagliptin or its salt and one or more pharmaceutically acceptable excipient provide immediate response, minimization of side effect, improving bioavailability and achieving the desired drug blood level rapidly and provide better patient compliance in the treatment of type 2 diabetes mellitus.

### FIELD OF THE INVENTION

The present invention relates to a pharmaceutical composition comprising Linagliptin or its salts, its process of preparation and method of use thereof.

More particularly, the present invention relates to a novel composition for parenteral administration comprising Linagliptin or its salts, its process of preparation and its indication as an adjunct to diet and exercise to improve glycemic control in adults with type 2 diabetes mellitus.

### BACKGROUND OF THE INVENTION

Type 2 diabetes mellitus (T2DM) is a complex metabolic disease that is characterized by hyperglycemia and reduced insulin biosynthesis/secretion in the context of whole-body insulin resistance. A characteristic of the disease is the inability of insulin producing B-cells in the pancreas to cope with the increased secretory demand of insulin-resistant organs and tissue, which over time leads to a vicious cycle of progressive loss of glycemic control and B-cell dysfunction.

Dipeptidyl peptidase 4 Inhibitors (DPP-4 inhibitors) are a novel class of therapeutic agents being developed to improve glycaemic control by inhibiting DPP-4-mediated inactivation of incretins such as glucagon-like peptide-1.

Linagliptin is a xanthine-based inhibitor of dipeptidylpeptidase-4 (DPP-4). Linagliptin is 1H-Purine-2,6-dione, 8-[(3R)-3-amino-1-piperidinyl]-7-(2-butyn-1-yl)-3,7-dihydro-3-methyl-1-[(4-methyl-2 quinazolinyl)methyl]-. The structural formula is as follows:

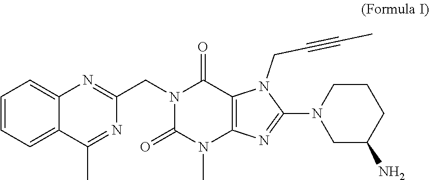 [SMILES: CC#CCn1c(N2CCC[C@@H](N)C2)nc2c1c(=O)n(Cc1nc(C)c3ccccc3n1)c(=O)n2C]

The empirical formula is C₂₅H₂₈N₈O₂. The molecular weight is 472.54 g/mol. Linagliptin is a white to yellowish, not or only slightly hygroscopic solid substance. It is very slightly soluble in water (0.9 mg/mL). Linagliptin is soluble in methanol (ca. 60 mg/mL), sparingly soluble in ethanol (ca. 10 mg/mL), very slightly soluble in isopropanol (<1 mg/mL), and very slightly soluble in acetone (ca. 1 mg/mL).

Linagliptin tablet oral was first approved by U.S. FDA on May 2, 2011 under the brand name TRADJENTA for Boehringer Ingelheim Pharmaceuticals Inc. it is available in 5 mg strength. The product is indicated for the treatment of type 2 diabetes mellitus in addition to diet and exercise.

U.S. Pat. No. 7,407,955 discloses a group of inhibitors of the dipeptidyl peptidase-4 (DPP-4) enzyme and claims Linagliptin and its physiologically acceptable salts.

U.S. Pat. No. 9,149,478 discloses a method for treating type 2 diabetes comprising subcutaneously or transdermally administering Linagliptin and basal insulin, wherein the administration of Linagliptin and basal insulin is carried out separately, sequentially or simultaneously, but U.S. Pat. No. 9,149,478 is not exemplified parenteral composition of Linagliptin or its salt.

U.S. Pat. No. 9,034,883 discloses method for treating oxidative stress, vascular stress and/or endothelial dysfunction in a sepsis patient comprising administering Linagliptin to the patient.

PCT publication no. WO2014009970A2 discloses an amorphous solid dispersion of Linagliptin in combination with a pharmaceutically acceptable carrier. Wherein the amorphous solid dispersion of Linagliptin is formulated into tablets, capsules, suspensions, dispersions or injectables. The said PCT application is more specifically related to preparation of amorphous linagliptin solid dispersion with carrier. Further, the PCT publication no. WO2014009970A2 is not exemplified parenteral composition of Linagliptin or its salt.

Linagliptin exhibits nonlinear pharmacokinetics after oral administrations which are mainly related to concentration-dependent binding of Linagliptin to the DPP-4.

As it is well known in the art that solid oral dosage forms have various limitations or disadvantages while manufacturing and using such as but not limited to low bioavailability, delayed onset of action, difficulty into administration to unconscious patient or the like.

According to the Biopharmaceutical classification system (BCS) Linagliptin classifies as a Class III compound i.e., it has high solubility and low permeability; it has incomplete oral systemic bioavailability which is approximately 30%.

Hence, applicant of the present invention has developed a novel composition for parenteral administration comprising Linagliptin or its salts, wherein said composition overcomes disadvantages associated with solid oral dosage forms and_also overcome the challenges of liquid as well as parenteral compositions.

It is a well-known fact that parenteral compositions should contain bare minimum excipients to avoid any possible side effects associated with the excipients and associated impurities in the excipient, since the compositions are introduced directly into the systemic circulation and bypasses the first pass metabolism, ultimately results in increased bioavailability which overcome the low bioavailability associated solid oral dosage form of Linagliptin or its salts.

The present invention attempts to provide a novel composition for parenteral administration comprising Linagliptin or its salts and at least one pharmaceutically acceptable excipient resulting in minimization of side-effects, immediate response, improving bioavailability, achieving the desired drug blood level rapidly and addresses the limitation associated with convention formulation(s).

### OBJECTS OF THE INVENTION

The main object of the present invention is to provide parenteral composition comprising:
a) Linagliptin or its salts; and
b) one or more pharmaceutically acceptable excipient.
   The main object of the present invention is to provide a parenteral composition comprising:
a) Linagliptin or its salts;
b) one or more solvent; and
c) one or more pharmaceutically acceptable excipient.
   Another object of the present invention is to provide a parenteral composition comprising:
a) 0.1-50% w/v of Linagliptin or its salts;
b) 01-90% w/v of solvent; and
c) q.s. of vehicle;
   Another object of the present invention is to provide a parenteral composition comprising:
a) 0.1-50% w/v of Linagliptin or its salts;
b) 1-90% w/v of solvent;
c) optionally q.s. of pH adjusting agent to adjust pH in a range of 7-8; and
d) q.s. of vehicle;
   Another object of the present invention is to provide a parenteral composition comprising:
a) 0.1-50% w/v of Linagliptin or its salts;
b) 01-90% w/v of solvent;
c) optionally 0.1-50% w/v of solubilizer;
d) optionally 0.1-30% w/v of surfactant;
e) optionally q.s. of pH adjusting agent to adjust pH in a range of 7-8; and
f) q.s. of vehicle;
   Another object of the present invention is to provide a parenteral composition comprising:
a) 0.1-50% w/v of Linagliptin or its salts;
b) 01-90% w/v of solvent;
c) 0.1-50% w/v of solubilizer;
d) 0.1-30% w/v of surfactant; and
e) q.s. of vehicle;

A further object of the invention is to provide a process for the preparation of a parenteral composition, comprising Linagliptin or its salts which comprises steps of:
1) Dissolving Linagliptin or its salts in weighed quantity of solvent with stirring for 15 minutes, continuously nitrogen sparging with maintaining of dissolved oxygen level below 1 ppm;
2) Adding vehicle to the above solution;
3) Checking the pH of the solution, adjust the pH of solution to about 7.4 with pH adjusting agent;
4) Making up the volume of Step 2 to 100ml with the vehicle;
5) Filtering the solution using sterile grade membrane filter PES 0.2 μ and fill in the 1 ml vial/ampoule with maintaining headspace oxygen level below 3% stoppering (Coated stopper) and sealing under laminar air flow (LAF).
   A further object of the invention is to provide a process for the preparation of a parenteral composition comprising Linagliptin or its salt which comprises steps of:
1) Dissolving Linagliptin or its salt in weighed quantity of solvent with stirring for 15 minutes, continuously nitrogen sparging with maintaining dissolved oxygen level below 1 ppm;
2) Mixing surfactant with a solubilizing agent;
3) Transferring the solution of Step 1 to the solution of Step 2;
4) Adding a vehicle to the above solution;
5) Checking pH of the solution, and adjusting the pH of solution to 7.4 with a pH adjusting agent;
6) Making up the volume of Step 2 to 100 ml with the vehicle;
7) Filtering the solution obtained in step 6) using sterile grade membrane filter PES 0.2 μ and fill in the 1ml vial/ampoule with maintaining the headspace oxygen level below 3% stoppering (Coated stopper) and sealing under laminar air flow (LAF).

A further object there is provided a parenteral composition of Linagliptin or its salt is indicated as an adjunct to diet and exercise to improve glycemic control in adults with type 2 diabetes mellitus.

### SUMMARY OF THE INVENTION

In one aspect, the present invention relates to a pharmaceutical composition comprising Linagliptin or its salts, in the form of a novel composition for parenteral administration.

In one aspect, the present invention relates to a novel composition for parenteral administration comprising Linagliptin or its salts and one or more suitable pharmaceutically acceptable excipient(s).

In another aspect, the present invention also relates to a process of preparation of a pharmaceutical composition comprising Linagliptin or its salts.

In another aspect, the present invention particularly relates to a pharmaceutical composition of Linagliptin or its salts in the form of a novel parenteral composition, its process of preparation and its indicated as an adjunct to diet and exercise to improve glycemic control in adults with type 2 diabetes mellitus.

In another aspect, the present invention provides a process for the preparation of a novel composition for parenteral administration comprising Linagliptin or its salts.

In another aspect, the present invention provides a method of using a novel composition for parenteral administration comprising Linagliptin or its salts is indicated as an adjunct to diet and exercise to improve glycemic control in adults with type 2 diabetes mellitus

In another aspect, the present invention provides a parenteral composition comprising:
a) Linagliptin or its salts; and
b) one or more pharmaceutically acceptable excipient.

In another aspect, the present invention provides a parenteral composition comprising:
a) Linagliptin or its salts;
b) one or more solvent; and
c) one or more pharmaceutically acceptable excipient.

In another aspect, the present invention provide a parenteral composition comprising:
a) 0.1-50% w/v of Linagliptin or its salts;
b) 01-90% w/v of solvent;
c) q.s. of pH adjusting agent to adjust pH in a range of 7-8; and
d) q.s. of vehicle;

In another aspect, the present invention provide a parenteral composition comprising:
a) 0.1-50% w/v of Linagliptin or its salts;
b) 01-90% w/v of solvent; and
c) q.s. of vehicle.

In another aspect, the present invention provides a parenteral composition comprising:
a) 0.1-50% w/v of Linagliptin or its salts;
b) 01-90% w/v of solvent;
c) 0.1-50% w/v of solubilizer;
d) 0.1-30% w/v of surfactant;
e) q.s. of pH adjusting agent to adjust pH in a range of 7-8; and
f) q.s. of vehicle.

In another aspect, the present invention provides a parenteral composition comprising:
a) 0.1-50% w/v of Linagliptin or its salts;
b) 01-90% w/v of solvent;
c) 0.1-50% w/v of solubilizer;
d) 0.1-30% w/v of surfactant; and
e) q.s. of vehicle.

In another aspect, the present invention provides a process for the preparation of a parenteral composition comprising Linagliptin or its salt which comprises steps of:
1) Dissolving Linagliptin or its salt in weighed quantity of solvent with stirring for 15 minutes, continuously nitrogen sparging with maintaining dissolved oxygen level below 1 ppm;
2) Adding a vehicle to the above solution;
3) Checking pH of the solution, and adjusting the pH of solution to 7.4 with a pH adjusting agent;
4) Making up the volume of Step 2 solution to 100 ml with the vehicle;
5) Filtering the solution using sterile grade membrane filter PES 0.2 μ and filling in the 1 ml vial/ampoule with maintaining the headspace oxygen level below 3% stoppering (Coated stopper) and sealing under laminar air flow (LAF).

In another aspect of an invention provides a process for the preparation of a parenteral composition, comprising Linagliptin or its salt which comprises steps of:
1) Dissolving Linagliptin or its salt in weighed quantity of solvent with stirring for 15 minutes, continuously nitrogen sparging with maintaining dissolved oxygen level below 1 ppm;
2) Mixing a surfactant with a solubilizing agent;
3) Transferring the solution of Step 1 to the solution of Step 2;
4) Adding a vehicle to the above solution of Step 3;
5) Checking pH of the solution, and adjusting the pH of solution to 7.4 with a pH adjusting agent;
6) Making up the volume of Step 2 to 100 ml with the vehicle;
7) Filtering the solution using sterile grade membrane filter PES 0.2 μ and filling in the 1 ml vial/ampoule with maintaining the headspace oxygen level below 3% stoppering (Coated stopper) and sealing under laminar air flow (LAF).

In another aspect of the present invention, the present invention provides a parenteral composition of Linagliptin or its salt for the treatment of Type 2 diabetes mellitus.

### DETAILED DESCRIPTION OF THE INVENTION

The term “composition” or “pharmaceutical composition” or “parenteral pharmaceutical composition” or “parenteral composition” or “novel composition for parenteral administration” as used herein synonymously include dosage forms such as solution, suspension, lyophilized powder, emulsion or the like.

The term “parenteral composition”, as in pharmaceutical composition, is intended to encompass a drug product comprising Linagliptin or its salts thereof and other inert ingredient(s) (pharmaceutically acceptable excipient(s). Such pharmaceutical compositions are synonyms with “formulation” and “dosage form”.

The term “comprising” is an inclusive term interpreted to mean containing, embracing, covering or including the elements listed following the term, but not excluding other unrecited elements.

The singular forms “a,” “an,” and “the” include the plurals unless the context clearly dictates otherwise.

The term “Linagliptin” is used in broad sense to include not only “Linagliptin” per se but also its pharmaceutically acceptable salts, solvates, hydrates, enantiomers, derivatives, isomers, polymorphs, prodrugs thereof and also its various crystalline and amorphous forms.

The amount of Linagliptin or its salts according to the invention may be present at 0.1 to 50% by weight based on total weight of the composition, more preferably 0.1 to 40% by weight based on total weight of the composition.

The term “pharmaceutically acceptable salt” or “salt thereof” refers to salts derived from a variety of organic and inorganic counter ions including hydrochloride, fumarate, maleate, phosphate, L-tartrate, citrate, acetate, oxalate, and sulfate or the like.

The term quantity sufficient (q.s.) refers to adding enough pH adjusting agent to adjust pH of the solution to a desired range.

The term “pharmaceutically acceptable salts” or “salt thereof” as used herein, includes those salts which are, within the scope of sound medical judgment, suitable for use in contact with the tissues of humans and lower animals without undue toxicity, irritation, allergic response and the like, which are well known in the art.

A “therapeutically effective amount” means the amount that, when administered to an animal for treating a disease, is sufficient to produce a desired therapeutic effect (e.g., to affect treatment for that disease).

The term “treating” or “treatment” refers to obtaining desired pharmacological and/or physiological effect. The effect can be therapeutic, which includes achieving, partially or substantially, one or more of the following results: partially or totally reducing the extent of the disease, disorder or syndrome; ameliorating or improving a clinical symptom or indicator associated with the disorder or delaying, inhibiting or decreasing the likelihood of the progression of the disease, disorder or syndrome.

“Pharmaceutically acceptable excipient(s)” are components that are added to the pharmaceutical composition other than the active ingredient Linagliptin. Excipients may be added to facilitate manufacture, enhance stability, enhance product characteristics, enhance patient acceptability etc. Pharmaceutically acceptable excipient(s) includes, but not limited to, solvent(s)/co-solvents, vehicle, buffer, antioxidant, preservative or antimicrobial agent, one or more lipid, emulsifying agent, surfactant, solubilizing agent, stabilizers, pH adjusting agent, chelating agent, acidifying agent, oily vehicle, suspending agent, dispersing agent, isotonicity adjusting agents and any other excipient known to the art for making pharmaceutical composition or combination thereof.

In one embodiment, the term “a novel parenteral composition” refers to a formulation comprising a therapeutically active ingredient and is suitable for injection into a patient such as a human in need thereof. In certain embodiments, a novel parenteral composition is a solution substantially sterile and does not contain any agents that are unduly toxic or infectious to the recipient.

In one embodiment, the composition of the invention has a pH in the range of 3-10, such as pH 6-10 or 6-9.

In one embodiment, the composition of the invention has a pH in the range of pH 6.5-8.5, such as pH 7.0-8.2 or 7.0-7.8

In one embodiment, the Linagliptin or its salts is present at a concentration of about 0.01 to 25 mg/mL, preferably about 0.01 to 10 mg/mL, preferably about 0.01 to 5 mg/mL, preferably about 0.01 to 1 mg/mL, preferably about 0.01 to 0.5 mg/mL, preferably about 0.01 to 0.2 mg/mL, preferably about 0.01 to 0.1 mg/mL and preferably about 0.01 to 0.05 mg/mL.

In one embodiment, a novel parenteral composition may be administered via parenteral route, such as injections, and include without limitation intravenous, intramuscular, intrapleural, intravascular, intrapericardial, intraarterial, intrathecal, intracapsular, intraorbital, intracardiac, intradennal, intraperitoneal, transtracheal, subcutaneous, subcuticular, intra articular, subcapsular, subarachnoid, intraspinal and intrastemal injection and infusion.

In one embodiment, the novel parenteral composition comprising Linagliptin or its salts is a solution for subcutaneous administration. In another embodiment, the novel parenteral composition comprising Linagliptin or its salts is a solution for intravenous administration.

The examples of vehicles include but not limited to water for injection, bacteriostatic water for injection, sterile water for injection, sodium chloride, lactated ringers, dextrose, peanut oil, cottonseed oil, corn oil, sesame oil, soybean oil, isopropyl myristate, ethyl oleate, benzyl benzoate, fixed oil, polyethylene glycol, propylene glycol, glycerin, sorbitol, ethyl alcohol, butylene glycol, polyethylene glycol, dioxolanes, dimethylacetamide, N-(β-hydroxyethyl)-lactamide, or the like or combinations thereof. Preferably the vehicle is water for injection. The vehicle according to present invention may be present in an amount to make up volume to quantity sufficient.

The examples of solvents include but not limited to sodium chloride, lactated ringers, dextrose, water for injection, sterile water for injection, bacteriostatic water for injection, water miscible solvents like dioxolanes, dimethylacetamide, N-(β-hydroxyethyl)-lactamide, butylene glycol, polyethylene glycol, propylene glycol, glycerin, ethyl alcohol(ethanol), water immiscible solvents like ethyl oleate, isopropyl myristate, benzyl benzoate, fixed oil, corn oil, cottonseed oil, peanut oil, sesame oil or the like or combinations thereof. The solvent according to present invention may be present in an amount from about 01-90% w/v by weight with respect to total volume of the composition. Preferably the solvent is ethanol.

The examples of co-solvents include but not limited to benzyl benzoate, ethyl oleate, isopropyl myristate, castor oil, cotton seed oil, N, N-dimethylacetamide, ethanol, ethanol dehydrated, dioxolanes, N-methyl 2-pyrrolidone, polyethylene glycol, propylene glycol, butylene glycol, glycerin, Glycofurol, sesame oil, soyabean oil, vegetable oil or the like or combinations thereof.

The examples of buffers include but not limited Sodium acetate trihydrate, phosphate (phosphoric acid), citric acid, succinic acid, histidine, glycine, arginine, malic acid, tartaric acid, acetic acid, benzoic acid, gluconic acid, glyceric, lactic acid, aconitic, adipic acid, ascorbic acid, fumaric acid, carbonic, glutamic acid, methionine, ammonium chloride, triethanolamine (tris) or the like or combinations thereof. The amount of buffering agent present in the composition ranges from about 0.001% to 10%; preferably about 0.01% to 5% based on the total volume of the composition.

The examples of antioxidants include but not limited to ascorbic acid, sodium metabisulfite, sodium bisulfite, sodium formaldehyde sulfoxylate, sodium ascorbate, thiourea, butylated hydroxyl anisole, butylated hydroxytoluene, ascorbic acid esters, monothioglycerol, propyl gallate, vitamin E, alpha-tocopherol or the like or combinations thereof. The amount of antioxidant present in the composition ranges from about 0.001% to 10%; preferably about 0.001% to 5%; more preferably about 0.001 to 2% based on the total volume of the composition.

The examples of preservative or antimicrobial agent include but not limited to parabens, benzalkonium chloride, benzethonium chloride, benzyl alcohol, chlorobutanol, m-cresol, Chlorocresol, myristyl gamma picolinium chloride, phenol, 2-phenoxyethanol, phenylmercuric nitrate, Thimerosal, phenylethyl alcohol, Sodium Benzoate, Sodium lactate, Sodium Sulfite, EDTA or the like or combinations thereof.

The examples of one or more lipid but not limited to soybean oil, fish oil, caprylic acid, capric acid, lauric acid, mysistic acid, palmitic acid, oleic acid, linoleic acid, α-linolenic acids, docosahexanoic acid (DHA) and eicosapentaenoic acid (EPA) or the like or combinations thereof.

The examples of emulsifying agent but not limited to Lecithin, Polyoxyethylene castor, Polyoxyethylene hydrogenated castor oil Base, Polyoxyethylene sorbitan monolaurate, sorbitan fatty acid esters, Acacia, Cetomacrogol, Cetyl alcohol, Glyceryl monostearate, Span 80, Polyoxyethylene stearate, Diethanolamine, Linoleic acid, Medium-chain Triglycerides, Sunflower Oil, Vitamin E Polyethylene Glycol Succinate, Lecithin, Polyoxyethylene polyoxypropylene copolymers (Pluronics) or the like or combinations thereof.

The examples of surfactants include but not limited to Polysorbate 20, Polysorbate 40, Polysorbate 60, Polysorbate 80, Polyoxyethylene sorbitan monooleate (Tween 80), Sorbitan monooleate, Polyoxyethylene sorbitan monolaurate (Tween 20), Lecithin, Polyoxyethylene polyoxypropylene copolymers (Pluronics) or the like or combinations thereof. Preferably the surfactant is Polysorbate 80. The surfactant according to present invention may be present in an amount from about 0.1-30% w/v by weight with respect to total volume of the composition.

The examples of solubilizer or solubilizing agent include but limited to Dimethylacetamide, Ethyl lactate, Glycerin, Lecithin, Polyoxyethylene Castor Oil Derivatives (PEG-40 castor oil), Polyethylene glycol, Polysorbate 20, Polysorbate 40, Polysorbate 60, Polysorbate 80, Povidone, Propylene glycol, Hydroxypropyl betadex, Macrogol 15 Hydroxystearate, Sorbitan monopalminate, Vitamin E Polyethylene Glycol Succinate or the like or combinations thereof. Preferably the solubilizer is Propylene glycol. The solubilizer according to present invention may be present in an amount from about 0.1-50% w/v by weight with respect to total volume of the composition.

The examples of stabilizers or stabilizing agents include but limited to Albumin, Asepsis sodium bicarbonate, L-Cysteine, Pentetic Acid (Diethylenetriamine pentaacetic acid), Ferric chloride, Hydrolyzed gelatin, D,L-Methionine, Potassium pyrosulfite, Potassium thiocyanate, Sodium gluconate and Zinc chloride solution, Lecithins or the like or combinations thereof.

The examples of pH adjusting agents according to present invention include but not limited to hydrochloric acid, citric acid, sulfuric acid, acetic acid, tartaric acid, lactic acid, Malic acid, Meglumine, Methionine, tromethamine, sodium hydroxide, potassium hydroxide, Sodium Phosphate Dibasic, Sodium Phosphate Monobasic, sodium carbonate or the like or combinations thereof. Preferably the pH adjusting agent is hydrochloric acid. The pH adjusting agent according to present invention may be present in an amount to achieve the desired pH.

The examples of chelating agents include but not limited to disodium edetate dihydrate, disodium edetate, edetic acid, ethylenediamine tertaacetic acid, calcium disodium ethylenediamine tertaacetic acid, diethylenetriamine pentaacetic acid, calcium versetamide sodium or the like or combinations thereof.

The examples of acidifying agent include but not limited to Citric acid, Acetic acid, Fumaric acid, Glacial Acetic Acid, Hydrochloric acid, Sulfuric acid, Nitric acid and Tartaric acid or the like or combinations thereof.

The examples of oily vehicle include but not limited to fixed oils, peanut oil, corn oil, cotton seed oil. Sesame oil, Soybean Oil, ethyl oleate, Isopropyl myristate, almond oil, Olive Oil or the like or combinations thereof.

The examples of suspending agent include but not limited to Carboxymethylcellulose Sodium, Methyl cellulose and polyvinyl pyrrolidone or the like or combinations thereof.

The examples of dispersing agent include but not limited to Oleic acid, Sodium pyrophosphate anhydrous, Lecithins, Poloxamer, Polyoxyethylene Sorbitan Fatty Acid Esters or the like or combinations thereof.

The examples of osmotic or tonicity adjusting agents include but not limited to sodium chloride, potassium chloride, calcium chloride, mannitol, glycerol, sorbitol, propylene glycol, dextrose, sucrose or the like or combinations thereof. The amount of osmotic or tonicity adjusting agents present in the composition ranges from about 0.001% to 10%; preferably about 0.01 to 5% based on the total volume of the composition.

In another aspect of the present invention is to provide process of manufacturing parenteral composition of Linagliptin or its salt along with one or more pharmaceutically acceptable excipients.

The process of preparing a parenteral composition of Linagliptin or its salt involves steps of:
1) Dissolving Linagliptin or its salt in weighed quantity of solvent with stirring for 10-20 minutes, continuously nitrogen sparging with maintaining dissolved oxygen level below 1 ppm;
2) Adding a vehicle to the above solution;
3) Checking pH of the solution, and adjusting the pH of solution to 7.4 with a pH adjusting agent;
4) Making up the volume of Step 2 to 100 ml with the vehicle;
5) Filtering the solution using sterile grade membrane filter PES 0.2 μ and filling in the 1 ml vial/ampoule with maintaining the headspace oxygen level below 3% stoppering (Coated stopper) and sealing under laminar air flow (LAF).

The process of manufacturing parenteral composition of Linagliptin or its salt involves steps of:
1) Dissolving Linagliptin or its salt in weighed quantity of solvent with stirring for 10-20 minutes, continuously nitrogen sparging with maintaining dissolved oxygen level below 1 ppm;
2) Mixing a surfactant with a solubilizing agent;
3) Transferring the solution of Step 1 to the solution of Step 2;
4) Adding a vehicle to the above solution;
5) Checking pH of the solution, and adjusting the pH of solution to 7.4 with a pH adjusting agent;
6) Making up the volume of Step 2 to 100 ml with the vehicle;
7) Filtering the solution of Step 6 using sterile grade membrane filter PES 0.2 μ and filling in the 1ml vial/ampoule with maintaining the headspace oxygen level below 3% stoppering (Coated stopper) and sealing under laminar air flow (LAF).

The concentration of Linagliptin or its salt, vehicle, cosolvant, optionally excipient and pH ranges with manufacturing process has been optimized in such way that parenteral composition of Linagliptin or its salt thereof according to present invention provide immediate response, minimization of side effect, improving bioavailability and achieving the desired drug blood level rapidly and provide better patient compliance and is indicated as an adjunct to diet and exercise to improve glycemic control in adults with type 2diabetes mellitus.

The parenteral composition of Linagliptin or its salt thereof according to present invention can be files into amber glass vial and seal using suitable rubber stopper or can be packaged in the ampoules, bottles or vials, bags and prefilled syringe.

The following example serves to illustrate the embodiments of the present invention. However, they do not intend to limit the scope of the invention. It is obvious to those skilled in the art to find out the composition for other dosage forms and substitute the equivalent excipients as described in this specification or with the one known in the pharmaceutical industry.

### EXAMPLES

The following Examples are provided solely for illustrative purposes and are not meant to limit the invention in any way.

### Formula 1: Linagliptin Injection 2 mg/mL Composition, 1 mL Vial/Ampoule

| Sr. | | Composition |
|---|---|---|
| No. | Ingredients | % (w/v) |
| 1. | Linagliptin | 0.2 |
| 2. | Ethanol | 20.00 |
| 3. | 0.1N Hydrochloric acid | q. s. to adjust pH |
| | solution | to 7.4 |
| 4. | Water for injection | q. s. to 100 mL |

### Manufacturing Process

1. Dissolving Linagliptin in weighed quantity of Ethanol with stirring for 15 minutes, continuously nitrogen sparging with maintaining dissolved oxygen level below 1 ppm;
2. Adding 60 mL of water for injection to the above solution;
3. Checking pH of the solution and adjusting the pH of solution to 7.4 with 0.1 N Hydrochloric acid solution;
4. Making up the volume of Step 2 to 100 mL with water for injection;
5. Filtering the solution using sterile grade membrane filter PES 0.2 μ and filling in the 1 ml vial/ampoule with maintaining the headspace oxygen level below 3% stoppering (Coated stopper) and sealing under LAF.
   Analytical Stability Data for 2 mg/mL in 1 mL Vial

| | | | 40° C./75% RH | | | | | 30° C./65% RH | | | 25° C./60% RH | | | |
|---|---|---|---|---|---|---|---|---|---|---|---|---|---|---|
| Sr. | | | 1 M | 2 M | 3 M | 6 M | 6 M | 3 M | 6 M | 6 M | 3 M | 6 M | 6 M | 9 M |
| No. | Parameters | Initial | UP | UP | UP | UP | IN | UP | UP | IN | UP | UP | IN | UP |
| Batch no. | | LINI-02-2109-012 | | | | | | | | | | | | |
| Pack | | 1 mL Amber vial, 13 mm FluroTec rubber stopper | | | | | | | | | | | | |
| 1. | Description | Clear colorless solution | | | | | | | | | | | | |
| 2. | pH | 7.70 | 7.91 | 7.74 | 7.95 | 7.65 | 7.70 | 7.92 | 7.62 | 7.66 | 7.96 | 7.68 | 7.70 | 7.53 |
| 3. | Assay of | 99.10 | 98.20 | 99.70 | 101 | 99.1 | 99.2 | 102.1 | 99.1 | 98.0 | 100.5 | 100.9 | 98.7 | 101.1 |
| | Linagliptin | | | | | | | | | | | | | |
| 4. | Related substances (%) | | | | | | | | | | | | | |
| 4.1 | N-Acetyl | 0.04 | 0.04 | 0.04 | 0.04 | 0.04 | 0.03 | 0.04 | 0.03 | 0.03 | 0.04 | 0.03 | 0.03 | 0.04 |
| 4.2 | Dimer | 0.08 | BLQ | 0.09 | 0.10 | 0.15 | 0.14 | 0.10 | 0.11 | 0.11 | 0.09 | 0.10 | 0.10 | 0.09 |
| 4.3 | N-BOC | BLQ | BLQ | BLQ | BLQ | BLQ | BLQ | BLQ | BLQ | BLQ | BLQ | BLQ | BLQ | BLQ |
| 4.4 | Single Maximum | BLQ | 0.08 | BLQ | BLQ | 0.04 | 0.03 | 0.10 | 0.04 | 0.04 | BLQ | 0.05 | 0.05 | 0.05 |
| | Unknown | | | | | | | | | | | | | |
| 4.5 | Total impurities | 0.12 | 0.12 | 0.13 | 0.14 | 0.23 | 0.20 | 0.24 | 0.18 | 0.18 | 0.13 | 0.18 | 0.18 | 0.28 |

### Formula 2: Linagliptin Injection 5 mg/mL Composition, 1 mL Vial/Ampoule

| Sr. | | Composition |
|---|---|---|
| No. | Ingredients | % (w/v) |
| 1. | Linagliptin | 0.5 |
| 2. | Ethanol | 20.00 |
| 3. | Propylene Glycol | 30.00 |
| 4. | Polysorbate 80 | 4.00 |
| 5. | 0.1N Hydrochloric acid | q. s. to adjust |
| | solution | pH to 7.4 |
| 6. | Water for injection | q. s. to 100 mL |

### Manufacturing Process

1. Dissolving Linagliptin in weighed quantity of Ethanol with stirring for 15 minutes, continuously nitrogen sparging with maintaining dissolved oxygen level below 1 ppm;
2. Mixing weighed quantity of Polysorbate 80 with weighed quantity of Propylene Glycol;
3. Transferring the solution of Step 1 to the solution of Step 2;
4. Adding 40mL of water for injection to the above solution;
5. Checking pH of the solution and adjusting the pH of solution to 7.4 with 0.1 N Hydrochloride acid solution;
6. Making up the volume of Step 2 to 100 mL with water for injection;
7. Filtering the solution using sterile grade membrane filter PES 0.2 μ and filling in the 1 ml vial/ampoule with maintaining the headspace oxygen level below 3% stoppering (Coated stopper) and sealing under LAF.
   Analytical Stability Data for 5 mg/mL

| | | | 40° C./75% RH | | | | | 30° C./65% RH | | | 25° C./60% RH | | |
|---|---|---|---|---|---|---|---|---|---|---|---|---|---|
| Sr. | | Initial | 1 M | 2 M | 3 M | 6 M | 6 M | 3 M | 6 M | 6 M | 3 M | 6 M | 6 M |
| No. | Parameters | Data | UP | UP | UP | UP | IN | UP | UP | IN | UP | UP | IN |
| Batch no. | | LINI-05-2110-014 | | | | | | | | | | | |
| Pack | | 1 mL Amber vial, 13 mm FluroTec rubber stopper | | | | | | | | | | | |
| 1. | Description | Clear colorless solution | | | | | | | | | | | |
| 2. | pH | 7.66 | 7.42 | 7.61 | 7.49 | 7.59 | 7.56 | 7.55 | 7.62 | 7.61 | 7.68 | 7.61 | 7.62 |
| 3. | Assay of | 97.9 | 101.3 | 102.1 | 99.5 | 98.0 | 100.7 | 99.9 | 99.7 | 98.4 | 98.2 | 99.8 | 101.4 |
| | Linagliptin | | | | | | | | | | | | |
| 4. | Related substances (%) | | | | | | | | | | | | |
| 4.1 | N-Acetyl | 0.07 | 0.11 | 0.08 | 0.07 | 0.06 | 0.07 | 0.06 | 0.06 | 0.06 | 0.07 | 0.07 | 0.07 |
| 4.2 | Dimer | 0.08 | 0.15 | 0.22 | 0.25 | 0.40 | 0.40 | 0.14 | 0.22 | 0.22 | 0.12 | 0.14 | 0.14 |
| 4.3 | N-BOC | BLQ | BLQ | BLQ | BLQ | BLQ | BLQ | BLQ | BLQ | BLQ | BLQ | BLQ | BLQ |
| 4.4 | Single Maximum | BLQ | 0.07 | BLQ | BLQ | 0.07 | 0.10 | 0.05 | 0.05 | 0.05 | 0.06 | 0.06 | 0.06 |
| | Unknown | | | | | | | | | | | | |
| 4.5 | Total impurities | 0.15 | 0.38 | 0.30 | 0.32 | 0.60 | 0.67 | 0.25 | 0.33 | 0.33 | 0.25 | 0.37 | 0.32 |

### Formula 3: Linagliptin Injection 0.5 mg/mL Composition, 1 mL Vial/Ampoule

| Sr. | | Composition |
|---|---|---|
| No. | Ingredients | % (w/v) |
| 1. | Linagliptin | 0.05 |
| 2. | Ethanol | 4.5 |
| 3. | 0.1N Hydrochloric acid | q. s. to pH to 7.4 |
| | solution | |
| 4. | Water for injection | q. s. to 100 mL |

### Manufacturing Process

1. Dissolving Linagliptin in weighed quantity of Ethanol with stirring for 15 minutes, continuously nitrogen sparging with maintaining dissolved oxygen level below 1 ppm;
2. Adding 60 mL of water for injection to the above solution;
3. Checking the pH of the solution, adjust the pH of solution to 7.4 with 0.1 N Hydrochloric acid solution;
4. Making up the volume of Step 2 to 100 mL with water for injection;
5. Filtering the solution using sterile grade membrane filter PES 0.2 μ and filling in the 1 ml vial/ampoule with maintaining the headspace oxygen level below 3% stoppering (Coated stopper) and sealing under LAF.

### Initial Analysis (Physical Evaluation) Data

| Sr. | | |
|---|---|---|
| No | Test Parameter | Results |
| 1 | Description | Clear colorless solution |
| 2 | pH | 7.45 |

### Formula 4: Linagliptin Injection 1 mg/mL Composition, 1 mL Vial/Ampoule

| Sr. | | Composition |
|---|---|---|
| No. | Ingredients | % (w/v) |
| 1. | Linagliptin | 0.1 |
| 2. | Ethanol | 4.5 |
| 3. | Propylene Glycol | 30.00 |
| 4. | Polysorbate 80 | 4.00 |
| 5. | 0.1N Hydrochloric acid | q. s. to pH |
| | solution | to 7.4 |
| 6. | Water for injection | q. s. to 100 mL |

### Manufacturing Process

1. Dissolving Linagliptin in weighed quantity of Ethanol with stirring for 15 minutes, continuously nitrogen sparging with maintaining dissolved oxygen level below 1 ppm.
2. Mixing weighed quantity of Polysorbate 80 with weighed quantity of Propylene Glycol;
3. Transferring the solution of Step 1 to the solution of Step 2;
4. Adding 40 mL of water for injection to the above solution;5. Checking pH of the solution and adjusting the pH of solution to 7.4 with 0.1 N Hydrochloride acid solution;
6. Making up the volume of Step 2 to 100 mL with water for injection;
7. Filtering the solution of Step 6 using sterile grade membrane filter PES 0.2 μ and filling in the 1 ml vial/ampoule with maintaining the headspace oxygen level below 3% stoppering (Coated stopper) and sealing under LAF.

### Initial Analysis (Physical Evaluation) Data

| Sr No | Test Parameter | Results |
|---|---|---|
| 1 | Description | Clear colorless solution |
| 2 | pH | 7.43 |

### Formula 5: Linagliptin Injection 2 mg/mL Composition, 1 mL Vial/Ampoule

| Sr. | | Composition |
|---|---|---|
| No. | Ingredients | % (w/v) |
| 1. | Linagliptin | 0.2 |
| 2. | Ethanol | 4.5 |
| 3. | Propylene Glycol | 30.00 |
| 4. | Polysorbate 80 | 4.00 |
| 5. | 0.1N Hydrochloric acid | q. s. to pH |
| | solution | to 7.4 |
| 6. | Water for injection | q. s. to 100 mL |

### Manufacturing Process

1. Dissolving Linagliptin in weighed quantity of Ethanol with stirring for 15 minutes, continuously nitrogen sparging with maintain dissolve oxygen level below 1 ppm;
2. Mixing weighed quantity of Polysorbate 80 with weighed quantity of Propylene Glycol;
3. Transferring the solution of Step 1 to the solution of Step 2;
4. Adding 40 mL of water for injection to the above solution;
5. Checking pH of the solutionand adjusting the pH of solution to 7.4 with 0.1 N Hydrochloride acid solution;
6. Making up the volume of Step 2 to 100 mL with water for injection;
7. Filtering the solution using sterile grade membrane filter PES 0.2 μ and filling in the 1 ml vial/ampoule with maintaining the headspace oxygen level below 3% stoppering (Coated stopper) and sealing under LAF.

### Initial Analysis (Physical Evaluation) Data

| Sr No | Test Parameter | Results |
|---|---|---|
| 1 | Description | Clear colorless solution |
| 2 | pH | 7.45 |

### Formula 6: Linagliptin Injection 5 mg/mL Composition, 1 mL Vial/Ampoule

| Sr. | | Composition |
|---|---|---|
| No. | Ingredients | % (w/v) |
| 1. | Linagliptin | 0.5 |
| 2. | Ethanol | 4.5 |
| 3. | Propylene Glycol | 30.00 |
| 4. | Polysorbate 80 | 4.00 |
| 5. | 0.1N Hydrochloric acid | q. s. to adjust |
| | solution | pH to 7.4 |
| 6. | Water for injection | q. s. to 100 mL |

### Manufacturing Process

1. Dissolving Linagliptin in weighed quantity of Ethanol with stirring for 15 minutes, continuously nitrogen sparging with maintaining dissolved oxygen level below 1 ppm;
2. Mixing weighed quantity of Polysorbate 80 with weighed quantity of Propylene Glycol;
3. Transferring the solution of Step 1 to the solution of Step 2;
4. Adding 40 mL of water for injection to the above solution;
5. Checking the pH of the solution, adjust the pH of solution to 7.4 with 0.1 N Hydrochloride acid solution;
6. Making up the volume of Step 2 to 100 mL with water for injection;
7. Filtering the solution using sterile grade membrane filter PES 0.2 μ and filling in the 1 ml vial/ampoule with maintaining the headspace oxygen level below 3% stoppering (Coated stopper) and sealing under LAF.

### Initial Analysis (Physical Evaluation) Data

| Sr No | Test Parameter | Results |
|---|---|---|
| 1 | Description | Clear colorless solution |
| 2 | pH | 7.44 |

1. A parenteral composition, comprising: a) Linagliptin or its salt; and b) one or more pharmaceutically acceptable excipient.

2. The parenteral composition as claimed in claim 1, wherein the Linagliptin or its salt is present in an amount of from about 0.1% to about 50% by weight with respect to total weight of the pharmaceutical composition.

3. The parenteral composition as claimed in claim 1, wherein the pharmaceutically acceptable excipient is selected from a group consisting of solvent(s), cosolvent, vehicle, buffer, antioxidant, preservative or antimicrobial agent, one or more lipid, emulsifying agent, surfactant, solubilizing agent or solubilizer, stabilizer, pH adjusting agent, chelating agent, acidifying agent, oily vehicle, suspending agent, dispersing agent, isotonicity adjusting agent and any combination thereof.

4. The parenteral composition as claimed in claim 1, comprising: a) 0.1-50% w/v of the Linagliptin or its salt; b) 01-90% w/v of the solvent; and c) q.s. of the vehicle.

5. The parenteral composition as claimed in claim 1, comprising: a) 0.1-50% w/v of the Linagliptin or its salt; b) 01-90% w/v of the solvent; c) optionally, 0.1-50% w/v of the solubilizer; d) optionally, 0.1-30% w/v of the surfactant; e) optionally, q.s. of the pH adjusting agent to adjust pH in a range of 7.0 to 7.8; and f) q.s. of the vehicle.

6. The parenteral composition as claimed in claim 1, wherein the parenteral composition is useful in treatment of type 2 diabetes mellitus in adults.

7. A process for preparation of a parenteral composition comprising Linagliptin or its salts, comprises steps of:
a) Dissolving Linagliptin or its salt in weighed quantity of solvent with stirring for 10-20 minutes, continuously nitrogen sparging with maintain dissolve oxygen level below 1 ppm;
b) Adding 01-90% w/v of a vehicle to the above solution;
c) Checking pH of the solution and adjusting the pH of solution to 7.4 with a pH adjusting agent;
d) Making up the volume of Step 2 to 100 ml with the vehicle;
e) Filtering the solution using a sterile grade membrane filter PES 0.2 p and filling in the 1 ml vial/ampoule with maintaining the headspace oxygen level below 3% stoppering (Coated stopper) and sealing under laminar air flow (LAF).

8. A process for preparation of a parenteral composition comprising Linagliptin or its salts, comprises steps of:
a) Dissolving Linagliptin or its salt in weighed quantity of solvent with stirring for 10-20 minutes, continuously nitrogen sparging with maintaining dissolved oxygen level below 1 ppm;
b) Mixing surfactant with solubilizing agent;
c) Transferring the solution of Step 1 to the solution of Step 2;
d) Adding 01-90% w/v of a vehicle to the above solution;
e) Checking pH of the solution and adjusting the pH of solution to 7.4 with a pH adjusting agent;
f) Making up the volume of Step 2 to 100 ml with the vehicle;
g) Filtering the solution using a sterile grade membrane filter PES 0.2 p and filling in the 1 ml vial/ampoule with maintaining the headspace oxygen level below 3% stoppering (Coated stopper) and sealing under laminar air flow (LAF).

9. The parenteral composition as claimed in claim 2, comprising: a) 0.1-50% w/v of the Linagliptin or its salt; b) 01-90% w/v of the solvent; and c) q.s. of the vehicle.

10. The parenteral composition as claimed in claim 3, comprising: a) 0.1-50% w/v of the Linagliptin or its salt; b) 01-90% w/v of the solvent; and c) q.s. of the vehicle.

11. The parenteral composition as claimed in claim 4, comprising: a) 0.1-50% w/v of the Linagliptin or its salt; b) 01-90% w/v of the solvent; c) optionally, 0.1-50% w/v of the solubilizer; d) optionally, 0.1-30% w/v of the surfactant; e) optionally, q.s. of the pH adjusting agent to adjust pH in a range of 7.0 to 7.8; and f) q.s. of the vehicle.
